(19) United States
(12) Patent Application Publication
(10) Pub. No.: US 2021/0116447 A1
(43) Pub. Date: Apr. 22, 2021

(54) LIQUID BIOPSY METHOD AND DEVICE

(71) Applicant: THE JOHNS HOPKINS UNIVERSITY, Baltimore, MD (US)

(72) Inventors: Dan Stoianovici, Reisterstown, MD (US); Doru Petrisor, Lutherville, MD (US); Changhan Jun, College Park, MD (US); Kenneth J. Pienta, Glen Arm, MD (US)

(21) Appl. No.: 16/971,228

(22) PCT Filed: Nov. 29, 2018
(86) PCT No.: PCT/US2018/062963

§ 371 Date: Aug. 19, 2020

Related U.S. Application Data
(60) Provisional application No. 62/632,219, filed on Feb. 19, 2018.

(51) Int. Cl. G01N 33/543; A61B 5/15; G01N 33/574; G01N 1/40; B03C 1/01; B03C 1/28

(52) U.S. Cl. CPC G01N 33/54333; A61B 5/150755; G01N 33/57492; B03C 2201/26; B03C 1/01; B03C 1/288; B03C 2201/18; G01N 1/4077

(57) ABSTRACT

An embodiment in accordance with the present invention provides a method and device that leverages a controlled scanning process for CTC-antibody binding, rather than classic random mixing. Magnetophoretic direct extraction is used to extract the CTCs onto a standard microscope slide. A device according to the present invention includes a vial into which a CTC bearing solution is combined with antibody-bound-PMP beads. An electromagnet is used to apply a magnetic field to engage the beads along the magnetic lines across the vial. The magnetic field and in turn the beads are moved on a fine pitch helical trajectory. The entire volume of the vial is scanned in this manner, allowing beads to come into close proximity to any CTCs present, and to collect all of the CTCs in the vial.

### CROSS REFERENCE TO RELATED APPLICATION

This application claims the benefit of U.S. Provisional Patent Application No. 62/632,219 filed on Feb. 19, 2018, which is incorporated by reference, herein, in its entirety.

### FIELD OF THE INVENTION

The present invention relates generally to medical devices. More particularly, the present invention relates to devices and methods for magnetic isolation and analysis of rare cells.

### BACKGROUND OF THE INVENTION

Circulating tumor cells (CTCs) are cancer cells that have escaped the local site of disease and entered into the peripheral blood. CTCs that survive the stress of the circulation and immune response can disseminate at distal sites (DTCs). As such, the capability to capture CTCs reliably from the blood for cytogenetic interrogations is critical for localized cancer and metastatic patients. CTCs biomarkers include a wide range of personalized medicine applications.

Counting CTCs is already being used to support therapeutic decisions and monitor the response to therapy in metastatic prostate, breast, and colorectal cancer. This can diminish adverse effects in patients who are not responding to therapy. Moreover, CTCs have the potential to properly determine the tumor-specific phenotypes that can guide personalized targeted therapies and make informed decisions regarding the choice of treatments effective against metastatic disease. Reliably capturing CTCs will also enable longitudinal measurements of CTCs as an indicator of disease burden and response to therapy. However, the isolation of these rare cells is difficult due to their very small concentrations, as low as 1:10⁹ of blood cells. Several techniques have been developed, but efficient isolation of CTCs as a ‘liquid biopsy’ continues to evade the scientific community.

Several methods have been developed to enumerate CTCs such as flow cytometry and high-throughput imaging. Cell isolation and retrieval methods include microfiltration size-based separation, density gradient centrifugation, and magnetophoretic separation.

To date, the majority of clinical work has been performed utilizing the CellSearch device, which uses the magnetophoretic separation principle. This was approved in 2004 and remains the only FDA approved device for CTC enumeration. Studies have shown that this device recovers CTCs from the blood of patients with metastatic cancer, in 20% to 70% of the patients. Another commercial device is the Celsee PREP 400 system, using a label-free microchannel size-based filtration approach.

The current challenge of liquid biopsy as a diagnostic tool is to improve the rates of CTC capturing, reduce CTC loss, and ultimately capture sufficiently many CTCs to be representative of the heterogeneity of the metastatic burden. This will require resolution at the single cell level, which no device can currently provide. Therefore, increasing the efficacy of CTC isolation and lossless retrieval of these rare cells is critical and remains a major technological challenge.

Current approaches use two types of separation methods: Physical methods and Immunocytochemical methods. Physical methods discriminate CTCs for separation based on size and density differences amongst cell types, with enrichment technologies such as filtration and centrifugation. These methods are associated with high CTC loss. Immunocytochemical methods discriminate the CTCs based on specific surface antigens that are only present on the CTCs but not on normal blood cells. Particular antibodies (EpCAM, epithelial cell adhesion molecule) are used to selectively bind to the antigens of the targeted CTCs based on their affinity. The antibody may be initially immobilized onto a collecting surface or bound to paramagnetic microparticles. In the case where cells are immobilized onto a collecting surface, the CTC bearing suspension is made to flow next to the collecting surface so that the antibody-antigen reactions may occur immobilizing the CTC onto the collecting surface. Their limitation is that it is then difficult to transfer the CTCs from the collecting surface without further loss.

In the case where cells are bound to paramagnetic micro-particles (PMPs), the antibody-bound-PMP (beads) are admixed with the CTC bearing suspension. Antibody-antigen reactions may occur if the beads come in contact with the CTCs by mixing, binding the CTCs and the beads. Manipulation and extraction of the bound CTCs is made by magnetic force acting on the PMPs (magnetophoresis).

Among other limitations, PMP based isolation commonly requires high bead concentrations to increase the rate of CTC isolation. Because the PMPs that remain unbound to CTCs are also extracted, this makes it difficult to visualize the CTCs, requiring further processing for separation and potential loss.

Therefore, it would be advantageous to provide a liquid biopsy method and device for fast, effective point-of-care results.

### SUMMARY OF THE INVENTION

The foregoing needs are met, to a great extent, by the present invention, wherein in one aspect a method for isolation of a rare cell population includes combining the rare cell population in a solution with antibody-bound-paramagnetic particles in a receptacle. The method includes applying a magnetic field to the combination of rare cell population and antibody-bound-paramagnetic particles, wherein the magnetic field is applied in a helical motion. The method includes moving the receptacle along a translation axis within the magnetic field. The method also includes binding the rare cell population to the antibody-bound-paramagnetic particles to generate rare cells bound to antibody-bound-paramagnetic particles.

In accordance with an aspect of the present invention the method includes transferring the rare cells bound to the antibody-bound-paramagnetic particles to a slide. The method includes isolating the rare cell population with no part of the rare cell population being discarded before the rare cells are extracted from the solution. The method also includes performing repeated tests on the same rare cell population. Further, the method includes applying an individual counting method of the rare cell population in a spiking experiment.

In accordance with another aspect of the present invention, a device for isolation of a rare cell population includes antibody-bound-paramagnetic microparticles. The device includes a receptacle configured to hold a solution of the rare cells and the antibody-bound-paramagnetic microparticles. The device includes an electromagnet configured to generate an electromagnetic field. Additionally, the method includes a movement mechanism coupled to the electromagnet and configured for moving the electromagnet and in turn the electromagnetic field about the receptacle, such that the antibody-bound-paramagnetic microparticles are brought into contact with the rare cells for binding to generate rare cells bound to antibody-bound-paramagnetic particle.

In accordance with yet another aspect of the present invention, the device includes a slide configured for receiving the rare cells. The device includes a magnet for facilitating transfer of the rare cells bound to the antibody-bound-paramagnetic particles. The movement mechanism has a helical motion and includes a mechanism for rotating the electromagnet and one that translates the vial along an axis. The device includes a video camera on the mechanism for rotating the electromagnet to provide a visualization during motion. In addition, the device includes a computing device with a non-transitory computer readable medium programmed for control of the movement mechanism.

In accordance with an aspect of the present invention, a device for isolation of a rare cell population includes a receptacle configured to hold a solution of the rare cells and rare cell binding paramagnetic microparticles. The device includes an electromagnet configured to generate an electromagnetic field. The device also includes a movement mechanism coupled to the electromagnets. The movement mechanism is configured for moving the electromagnets and in turn the electromagnetic field about the receptacle, such that the rare cell binding paramagnetic microparticles are brought into contact with the rare cells for binding to generate rare cells bound to the rare cell binding paramagnetic particles, in a scanning motion process configured to sweep the contents of the entire receptacle.

In accordance with yet another aspect of the present invention, the device includes a microscope slide configured for receiving the rare cells. The device includes a magnet for facilitating transfer of the rare cells bound to the rare cell binding paramagnetic particles. The movement mechanism has a helical motion of the magnetic field relative to the receptacle. The movement mechanism further includes a mechanism for rotating the electromagnet and one that translates the receptacle along an axis. The device further includes a video camera in a fixed location relative to the magnetic field, to provide visualization during motion. The device also includes a computing device with a non-transitory computer readable medium programmed for control of the device.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates a perspective view of a magnetic scanning device of the present invention, and FIG. 1B illustrates the transfer of the CTC to a slide.

FIGS. 2A-2H illustrate views of the device assembly.

FIG. 3 illustrates a perspective view of a CTC-X device.

FIG. 4A illustrates a top view of electromagnet and helical scan cycles, and FIG. 4B illustrates a side view of a vial during an electromagnet and helical scan cycle.

FIGS. 5A-5I illustrate schematic views of direct extraction of the beads and bound CTCs to the microscope slide.

FIGS. 6A and 6B illustrate exemplary image views of Cells IN and Cells OUT from the same experiment.

FIG. 7 illustrates a graphical view of reported device results compared to previous results from the ST device.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a method and device for using a controlled process to maximize the opportunities for binding of each CTC in the solution with beads, even if the beads are in relatively low concentrations. The method and device leverage a controlled scanning process for CTC-antibody binding, rather than classic random mixing. Magnetophoretic direct extraction is used to extract the CTCs onto a standard microscope slide. A device according to the present invention includes a vial into which a CTC bearing solution is combined with antibody-bound-PMP beads. An electromagnet is used to apply a magnetic field to engage the beads along the magnetic lines across the vial. The magnetic field and in turn the beads are moved on a fine pitch helical trajectory. The entire volume of the vial is scanned in this manner, allowing beads to come into close proximity to any CTCs present, and to collect all of the CTCs in the vial.

The most promising techniques for CTC isolation thus far have been immunocytochemical. These immunocytochemical techniques label CTCs for separation based on surface antigens that bind selectively to CTCs, distinguishing them from the normal bystander cells. A PMP type was chosen for its potential to further transfer the CTCs for the evaluation onto a standard microscope slide.

With all current immunocytochemical magnetophoretic CTC isolation devices:
1. Sample loss is difficult to quantify and control. Part of the CTCs bearing suspension originating from the whole blood is typically discarded, either in the preprocessing steps or within the isolation device. The related CTCs loss remains unknown.
2. The process of CTCs-antibody binding relies primarily on random mixing methods such as shaking, rocking, tumbling, centrifugal, microvortex, and incubation. If a part of the CTCs remain unbound, these will not be isolated by magnetophoresis and be lost.

CTC loss may lead to a misrepresentation of the heterogeneity of the metastatic burden within the liquid biopsy and impacts directly the rate of CTC recovery. This is not yet optimal with any current device, for example up to 82% with the CellSearch, 80% with the VerIFAST device, and nearly 85% with the ST device. Two possible causes of this effect are that:
1. The antibody-antigen reactions occur for most CTCs and most are bound to the respective PMPs, but the magnetophoresis is insufficiently strong to extract them from the suspension.
2. And/or, antibody-antigen reactions fail to occur for numerous CTCs.

The common approach to the low recovery problem has been to use higher concentrations of antibody-labeled beads, which has indeed translated into higher CTC recovery rates. But even so, recovery remains still short of 100%. Moreover, at these high concentrations, numerous PMPs cluster on a CTC, and numerous unbound PMPs are extracted magnetically from the solution. These extra PMPs impede microscopic visualization and require further processing to separate the PMPs, which is susceptible to additional CTC loss. Therefore, using high bead concentrations is not ideal.

With current devices, the process of antibody-antigen binding is random. Typically, the antibody-PMP beads are admixed within the CTC bearing suspension and tumbled. In order for the binding to occur, a CTC must come in direct contact with a bead in order to engage the antibody-antigen reaction. Cell binding can be conceptualized as the number of opportunities for binding afforded for each CTC in the solution to come in direct contact with one or more beads, multiplied by the probability of each opportunity to result in actual binding. In the classic methods, the likelihood of binding events occurring randomly is directly proportional with the number of PMPs and beads. Since the CTCs are scarce, a very high number of beads must be used to capture them.

Previous experiments that use tumble binding showed that spiked CTC recovery was noticeably dependent on bead concentration, suggesting that missed bindings represent an important cause of low CTC recovery. Immunocytochemical selective binding to CTCs and magnetophoretic manipulation methods are used in conjunction with the present invention. The novel method of the present invention circumvents the common random process of CTC-bead antibody binding by using a controlled process to maximize the opportunities for binding of each CTC in the solution with beads, even if the beads are in relatively low concentrations.

Relative to the previous methods the device incorporates two novel techniques:
1. A controlled scanning process for CTC-antibody binding, rather than the classic random mixing. The hypothesis is that this will yield high CTCs recovery with relatively low bead concentrations.
2. A novel magnetophoretic direct extraction method to extract the CTCs onto a standard microscope slide. The hypothesis is that no part of the original CTCs bearing suspension has to be discarded prior to CTCs extraction.
   The combination of these 2 methods has the potential to render a lossless CTCs isolation device.

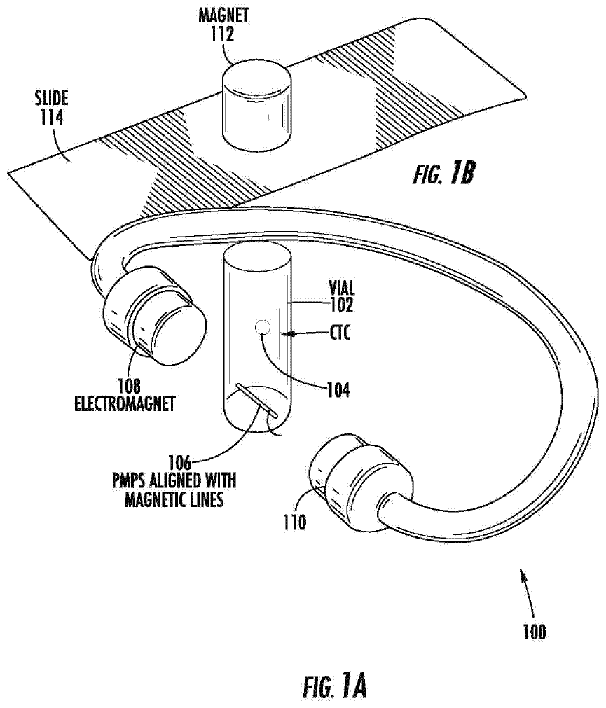

The operating principle of the device is illustrated in FIGS. 1A and 1B. FIG. 1A illustrates a perspective view of a magnetic scanning device of the present invention, and FIG. 1B illustrates the transfer of the CTC to a slide. FIG. 1A illustrates a device 100, according to an embodiment of the present invention. The device 100 includes a vial 102. The vial 102 is configured to hold a solution containing CTCs 104. Only 1 CTC 102 is shown in FIG. 1A for simplicity. The CTCs bearing solution is placed in the vial 102 together with the antibody-bound-PMPs (beads) 106. As illustrated in FIG. 1A, an electromagnetic field, generated by electromagnets 108, 110, engages the beads 106 along a magnetic line across the vial 102. A mechanism, described further herein, moves the magnetic field and beads 106 on a fine pitch helical trajectory. The pitch is smaller than the width of the bead strip, so that the strip scans the volume of the vial during helical motion. By scanning the entire volume of the CTC bearing suspension, beads come into close proximity of any CTC allowing them to initiate the binding reaction. The process can be repeated with other scans, which should bind and collect ideally all CTCs in the vial.

Finally, the beads 106 and bound CTCs 104 are extracted from the top with another magnet 112, as illustrated in FIG. 1B. For this process, the vial 102 is filled so that the slide 114 comes in direct contact with the suspension. At this time the electromagnets 108, 110 are switched off passing the PMPs to the magnet 112, and transferring the CTCs 102 onto the slide 114 as shown in FIG. 1B.

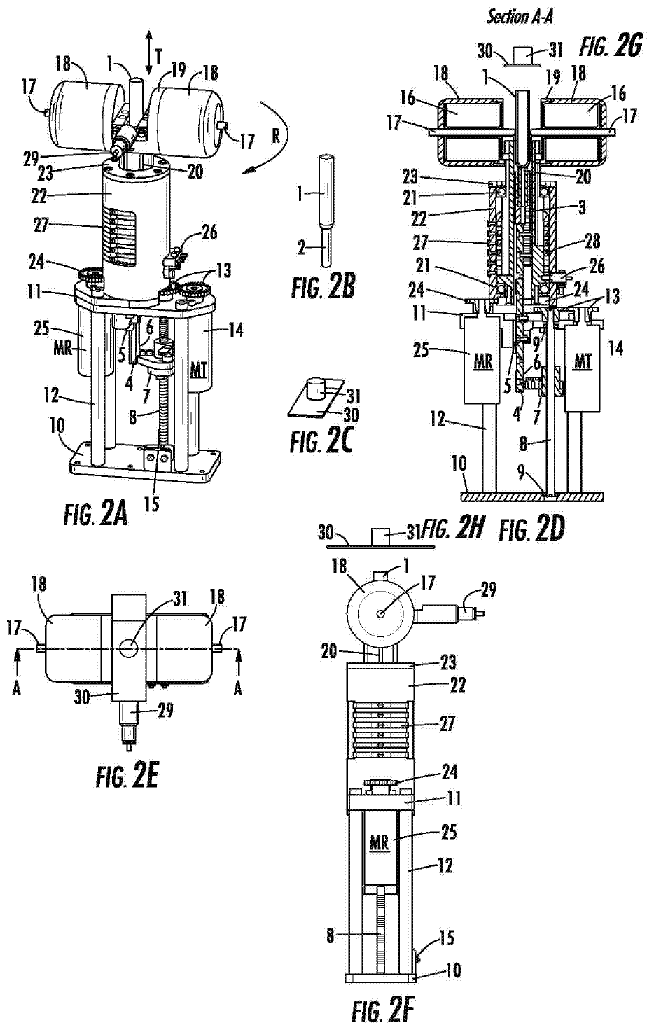

A new CTC extraction device (CTC-X) was developed based on the principle described above. The device was designed and analyzed using Creo Parametric CAD software. FIG. 2A-2H illustrate views of the device assembly. The helical motion of the magnetic field is implemented by two mechanisms, one that rotates the electromagnets (R) and one that translates (T) the vial.

With respect to the translational axis (T), illustrated in FIGS. 2A, 2D, and 2F, the vial (1) presents a common shape but includes an extension (2) that allows it to be easily and precisely positioned within the device. The vial is inserted into a support (3) that is attached to a linear rail (4), riding on linear guides, (5) attached to an upper plate base (11). The rail is connected elastically (6) (to prevent vibrations from the motors to dissipate to the vial) to a ball-nut (7), driven by a ball-screw (8). The screw (8) is supported at both ends by bearings (9) between the lower (10) and upper (11) plates. The plates are connected by spacer bars (12), and together form the base of the assembly. The screw is engaged by a pair of spur gears (13), driven by an MT motor (14). The motor includes incremental position encoders. A limit switch (15) is used to home this axis.

With respect to the rotational axis (R), shown in FIGS. 2A and 2D-2F, the electromagnets are made by coils (16) and cores (17) that can be advanced or retracted within. Together with their caps (18), the electromagnets are attached to a central hub (19), mounted on a shaft (20), supported by bearings (21) into a case (22) and cap (23). The hub assembly is rotated from a motor MR (25) through a pair of spur gears (24). Because the encoders of the motor are incremental, a limit switch (26) is used as well for this axis. A video camera is included on the rotary assembly at the level and normal to the magnetic field lines, for a close-up visualization during motion. Electrical circuits to the rotary assembly are made through custom-made slip rings, including brushes (27) and rings (28). Channels made in the shaft (20) and hub (19) allow electrical wires to be pulled internally from the rings to the coils and the camera. The slide (30) is a standard microscope slide. This and the permanent magnet (31) are operated manually.

FIG. 2B illustrates a side view of a collection vial (1) for the solution of CTCs and PMPs. The vial (1) can also include a base (2) for coupling the vial to the device. FIGS. 2C, 2G, and 2H illustrate perspective and side views of a slide (30) and a secondary magnet (31) for transferring the bound-CTCs to the slide. The process of transferring the bound-CTCs to the slide will be described in further detail below.

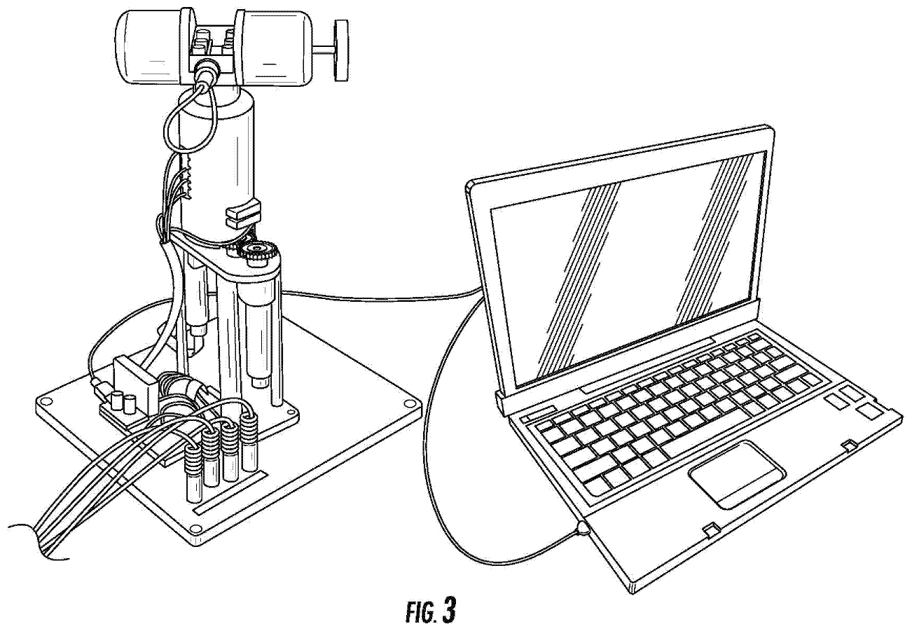

A photograph of the device is presented in FIG. 3. FIG. 3 illustrates a perspective view of a CTC-X device. The device is controlled by a custom electronic board controlled by a laptop or PC over the USB, and is powered by DC power supplies. The board uses EPOS4 24-1.5 (Maxon Motor, Switzerland) digital positioning controller modules for motion control of the motors (Maxon 203187: 12V, 29:1 gearhead, 16 ct encoder) and to control the coils through solid state relays (70M-ODC, Grayhill Inc., La Grange, Ill.).

Custom electromagnets were built with the following specifications: 28 AWG magnet wire, 2000 turns, soft iron 5.1 mm 42 mm length core. A program was written in Visual C++ (Microsoft) to control the system. Coordinated motion of the 2 motorized axes is used to generate helical trajectories. The activation of the electromagnets is coordinated with the rotary axis. Control parameters can be adjusted in software as needed.

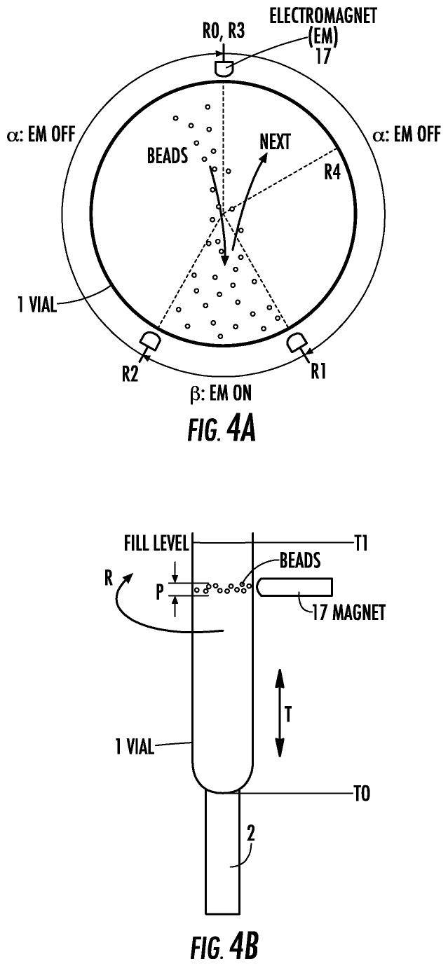

FIGS. 4A and 4B illustrate electromagnet and helical scan cycles. FIG. 4A illustrates a top view of electromagnet and helical scan cycles, and FIG. 4B illustrates a side view of a vial during an electromagnet and helical scan cycle. Various schemes to coordinate the magnetic field with its rotation can be implemented by the device. A cycle to cloud the beads in suspension across the surface of the vial at the level of the magnet cores is shown in FIG. 4A. Starting from a position (R0), the magnet is first turned off during the time it takes to rotate with an angle α<180° to position (R1), and then switched on for an angle β up to position (R2). The cycle repeats, for example in the next step the magnet is off to position (R3), and then on to position (R4), and so on. Therefore, the magnetic field from 1 or 2 electromagnets is turned on/off in certain cyclical patterns. The two angles are set so that:

2α+β=360° (Eq. 1)

Angle α is set experimentally, together with the duration of magnet activation t[s] that it takes to rotate with angle β. The angular velocity of axis R is then calculated as:

$$ω = \frac{β}{t} = \frac{2 α - 360}{t} [ ° \text{/} s ] \qquad ( Eq . 2 )$$

This cycle uses only one magnet, but may be extrapolated to multiple magnets, and may potentially eliminate the need for the rotation axis.

The translation axis (T) moves the vial up and down within the rotating magnetic field (R). The translation (T) is coordinated with the rotation (R) to implement a helical trajectory. The pitch p of the helical trajectory is set experimentally, so that is smaller than the thickness of the bead cloud at the level of the magnets (p<P). The process is illustrated in FIG. 4b.

The helical motion scans the volume of the vial with a cloud of beads. The process normally starts from the bottom of the vial (T0) and continues to the top (T1), at the level at which the vial was filled. Multiple helical bottom-top and top-bottom passes are done to facilitate the cells to bind to the scanning cloud of beads. The final pass must end at the top end to allow the beads and bound cells to be extracted, as follows.

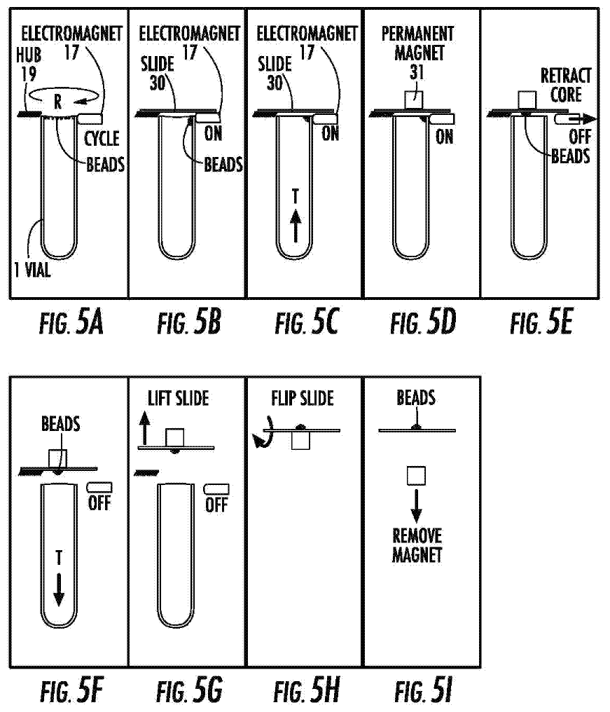

The device was designed to allow the direct transfer (extraction) of the beads and bound cells to a standard microscope slide, as illustrated in FIGS. 5A-5I. FIGS. 5A-5I illustrate schematic views of direct extraction of the beads and bound CTCs to the microscope slide. The transfer involves the following steps:

1) End scan cycle (illustrated in FIG. 5A): As shown above, the helical scan ends at the top of the vial. The beads and bound CTCs are distributed across the vial at the level of the electromagnet.

2) Stop scan and place microscope slide (illustrated in FIG. 5B): The translational motion is stopped, the electromagnet is turned ON, and the rotation stops to a home position. The beads and bound CTCs are pulled on the vial wall, on the side of the electromagnet. If not already done so, fill the vial to the very top so that the liquid bubbles above the edge (convex shape). Place the microscope slide face-down above the vial resting it on the hub of the device (19). At this vial position the slide is slightly above the top of the liquid in the vial.

3) FIG. 5C illustrates the next step to slowly raise the vial with the translation axis (T) so that the liquid touches the slide. A small region of liquid contact is visible through the slide.

4) FIG. 5D illustrates the step of placing a permanent magnet on the slide above the liquid contact region.

5) FIG. 5E illustrates the step of stopping the electromagnet and slightly pulling its core out. The beads and bound CTC are pulled through the liquid on the side of the slide.

FIG. 5F illustrates the step of slowly lowering the vial with the translation axis (T). A small bubble of liquid remains on the slide together with the beads and bound CTC.

FIG. 5G illustrates the step of carefully lifting the slide together with the magnet.

FIG. 5H illustrates the step of holding the magnet onto the slide, and carefully flip the slide on the other side.

FIG. 5I illustrates the step of removing the magnet puling it down from the slide. A small bubble of the beads and CTC is available for visualization and further processing on top of the slide.

Initial experiments with the device were performed in order to show the functionality of the present invention. The experiments and examples discussed herein are not meant to be considered limiting and are include merely to further illustrate the present invention. The recovery rates of CTC in spiking experiments was measured. Few cells were initially counted, diluted in solution, ran through the device, extracted to slide, and finally recounted to determine how many cells are recovered. Accurate counting the CTC in and out of the experiments is critical to determine the efficacy of the device.

CTC recovery experiments commonly use an average counting method that relives on the approximate number of cells per volume unit. When applied to counting a few cells in a small volume (for example 50 cells/μL or lower) errors are large. Instead, the actual CTCs that went into each experiment were counted exactly. For this, the vial of the device (1, 2 assembly) was removed from the device. A small volume of a much diluted CTC bearing suspension was pipetted inside, on a spot on the lateral side of the vial. The vial was placed on its side on a microscope, with the CTC spot pointing towards the lenses, and imaged. All cells were exactly counted.

A vial with 5.8 mm inner diameter and a 12.6 mm height was used (calculated volume 307 μL). PC3 line prostate cancer cells with green fluorescent protein (GFP) expression were prepared in a diluted input cells suspension (approx. 50 cells/μL). Over the experiments the suspension was further diluted with phosphate buffered saline (PBS) solution to reduce the number of cells (approx. 2.4 cells/μL). Before each experiment, the suspension was mixed in a shaker. A volume that corresponds to either 5 or 25 average counted cells was pipetted on spot on the wall of the vial (Cells IN—Average). The vial was then placed on the microscope, cells were counted individually, and recorded as Count IN—Individual. Without delay (to prevent drying), the vial was filled with a phosphate buffered saline (PBS) solution to cover the CTC drop spot. Then, 1 μL of Epithelial Enrich DynaBeads (ThermoFisher Scientific, Waltham, Mass.) (4*10⁵ beads/μL) was added to the vial. Finally, the vial was filled to the top level with PBS to a total of 0.3mL.

The following parameters were used: Electromagnet power 20V, 63 bottom-top helical scan cycles, pitch p=0.5 mm, scan stroke 11 mm, α=150°, and t=0.4 s. The total duration of the scan cycle was 44 min. After the scan, the vial was further filled with as few μL of PBS as needed to facilitate the transfer to the slide. The beads and bound CTCs were transferred to the microscope slide, imaged, and counted (Count OUT) without delay to prevent drying on the slide. The experiment was repeated 12 times.

Cell recovery rate was calculated as:

$$ℛ = \frac{Cells OUT}{Cells IN} * 100 [ \% ] \qquad ( Eq . 3 )$$

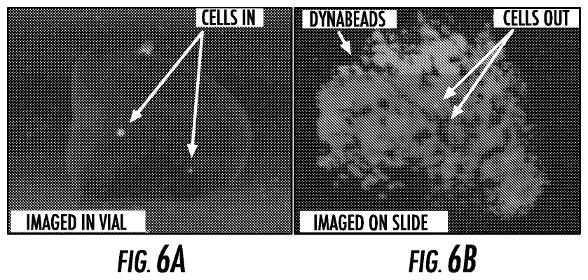

An example, from experiment number 3, of a microscope image taken within the vial for the Cells IN count, and image of the beads and CTS on the microscope slide together with the Cells OUT count is shown in FIGS. 6A and 6B. FIGS. 6A and 6B illustrate exemplary image views of Cells IN and Cells OUT from the same experiment.

The numbers of Cells IN measured with the average as well as individual count methods are shown in Table 2. This also shows the Cells OUT numbers, counted individually, as well as the cell recovery rates calculated based on the average as well as individual count methods.

The results of the experiments are shown in Table 1. The CTC recovery rate in PBS spiked experiments was 100%.

TABLE 1
Experimental Results

| Experiment Number | | 1 | 2 | 3 | 4 | 5 | 6 | 7 |
|---|---|---|---|---|---|---|---|---|
| Cells IN | Average Count | 5 | 5 | 5 | 25 | 25 | 25 | 5 |
| | Individually | 8 | 5 | 2 | 20 | 12 | 15 | 1 |
| | Counted | | | | | | | |
| Cells OUT | Individually | 8 | 5 | 2 | 20 | * | 15 | 1 |
| | Counted | | | | | | | |
| Cell | Average Count | 32% | 100% | 40% | 80% | * | 60% | 20% |
| Recovery | Individual | 100 | 100 | 100 | 100 | * | 100 | 100 |
| Rate [symbol] [%] | Count | | | | | | | |

| Experiment Number | | 8 | 9 | 10 | 11 | 12 | Average |
|---|---|---|---|---|---|---|---|
| Cells IN | Average Count | 5 | 5 | 5 | 5 | 5 | — |
| | Individually | 4 | 4 | 2 | 1 | 2 | — |
| | Counted | | | | | | |
| Cells OUT | Individually | 4 | 4 | 2 | 1 | 2 | — |
| | Counted | | | | | | |
| Cell | Average Count | 80% | 80% | 40% | 20% | 40% | 54% |
| Recovery | | | | | | | |
| Rate [symbol] | Individual | 100 | 100 | 100 | 100 | 100 | 100% |
| [%] | Count | | | | | | |

Note:
* Count OUT lost due to imaging delays

Table 1 shows that large variability exists between the cells counted with an average based method and their actual, individually counted numbers for the Cells IN count. This reveals the importance of the individual count that was derived. Without it, recovery rate would have varied in the experiments between 20% -100%, being unreliable, disorienting for device setup, and most likely fail device developments. The data argues strongly against average-based cell counting for low sample volumes of cells in low density suspension.

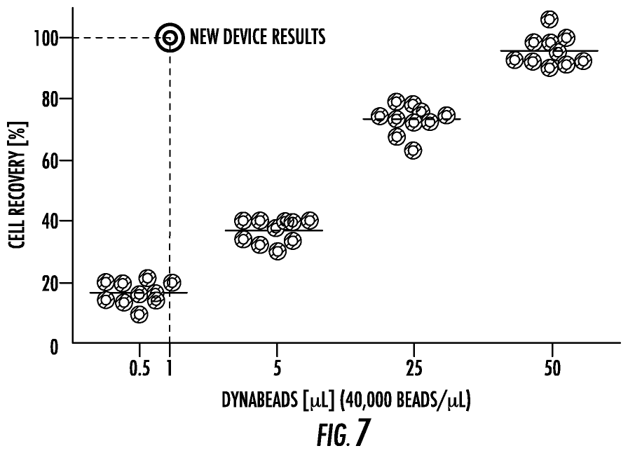

Preliminary experiment results are superior to previous devices. For example, FIG. 7 presents the current results together with results from previous devices, such as the ST device. There, the number of beads had to be increased progressively to increase the recovery rates. By contrast, the new device achieves 100% recovery with 50 times fewer beads. FIG. 7 illustrates a graphical view of reported device results compared to previous results from the ST device. Average counting was used in the previous experiment. FIG. 7 shows >100% recovery rates that can only be explained by counting errors. This is another example in support of the individual counting method presented.

A novel CTC isolation device was developed. The CTC recovery rate in initial CTC-spiked experiments was 100%. This supports the hypotheses of the study that high yield CTC recovery can be done with relatively low bead concentrations. No part of the original CTCs bearing suspension has to be discarded prior to CTCs extraction. The new device offers a unique potentially lossless CTCs isolation solution. In turn, this offers the ability of the device to be used on repeat tests on the same CTC bearing solution, for similar tests to check for missed CTCs, or tests with different antibodies.

Additional tests are required to validate the device. Immediately following tests will be the recovery of spiked cancer cells in blood. The experiments will be performed similarly but using blood instead of PBS.

The initial tests and application of the device will be for epithelial prostate cancer CTC. However, the device is readily usable for other type of rare cell isolation in other bodily fluids. Potential applications include rare cells such as circulating stem cells, circulating endothelial cells for vascular and cardiology research, and circulating placenta derived cells in gynecology and obstetrics. Additional application beyond the liquid biopsy from blood include the use of the device in evaluating bone marrow and other body fluids such as urine, saliva, semen, and cerebro-spinal fluid (CSF), cytology specimens from fine-needle aspiration (FNA), etc.

The present invention offers advantages over prior devices for cell isolation and analysis. The device of the present invention presents a novel construction with a horizontal rather than a vertical orientation making it possible to use larger capacity wells. Moreover, the integration of the drain well and syringe facilitates downstream processing on the device of the present invention. Most importantly, the sieve may be removed from the device for imaging on a standard microscope slide.

The control of the present invention can be carried out using a computer, non-transitory computer readable medium, or alternately a computing device or non-transitory computer readable medium incorporated into the robotic device. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The operating console for the device is a non-generic computer specifically designed by the manufacturer. It is not a standard business or personal computer that can be purchased at a local store. Additionally, the console computer can carry out communications through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method for isolation of a rare cell population comprising:
combining the rare cell population in a solution with antibody-bound-paramagnetic particles in a receptacle;
applying a magnetic field to the combination of the rare cell population and the antibody-bound-paramagnetic particles, wherein the magnetic field is applied in a cyclical pattern;
moving the magnetic field relative to the receptacle along a translation axis; and
moving the magnetic field relative to the receptacle about a rotation axis; and
binding the rare cell population to the antibody-bound-paramagnetic particles to generate rare cells bound to antibody-bound-paramagnetic particles.

2. The method of claim 1 wherein the magnetic field is applied in a generally helical motion.

3. The method of claim 1 further comprising transferring the rare cells bound to the antibody-bound-paramagnetic particles to a microscope slide.

4. The method of claim 1 further comprising isolating the rare cell population with no part of the rare cell population being discarded before the rare cells are extracted from the solution.

5. The method of claim 1 further comprising performing repeated tests with the same or different antibody on the same rare cell population.

6. The method of claim 1 further comprising applying an individual counting method of the rare cell population in a spiking experiment.

7. A system for isolation of a rare cell population comprising:
antibody-bound-paramagnetic microparticles;
a receptacle configured to hold a solution of the rare cells and the antibody-bound-paramagnetic microparticles;
several electromagnets configured to generate an electromagnetic field; and,
a movement mechanism coupled to the electromagnets and configured for moving the electromagnets and in turn the electromagnetic field about the receptacle, such that the antibody-bound-paramagnetic microparticles are brought into contact with the rare cells for binding to generate rare cells bound to antibody-bound-paramagnetic particles, in a scanning motion process configured to sweep the contents of the entire receptacle.

8. The system of claim 7 further comprising a microscope slide configured for receiving the rare cells.

9. The system of claim 8 further comprising a magnet for facilitating transfer of the rare cells bound to the antibody-bound-paramagnetic particles.

10. The system of claim 7 further comprising the movement mechanism having a helical motion of the magnetic field relative to the receptacle.

11. The system of claim 7 wherein the movement mechanism further comprises a mechanism for rotating the electromagnets and one that translates the receptacle along an axis.

12. The system of claim 11 further comprising a video camera in a fixed location relative to the magnetic field, to provide visualization during motion.

13. The system of claim 7 further comprising a computing device with a non-transitory computer readable medium programmed for control of the device.

14. A device for isolation of a rare cell population comprising:
a receptacle configured to hold a solution of the rare cells and rare cell binding paramagnetic microparticles;
an electromagnet configured to generate an electromagnetic field; and,
a movement mechanism coupled to the electromagnet and configured for moving the electromagnet and in turn the electromagnetic field about the receptacle, such that the rare cell binding paramagnetic microparticles are brought into contact with the rare cells for binding to generate rare cells bound to the rare cell binding paramagnetic particles, in a scanning motion process configured to sweep the contents of the entire receptacle.

15. The device of claim 14 further comprising a microscope slide configured for receiving the rare cells.

16. The device of claim 15 further comprising a magnet for facilitating transfer of the rare cells bound to the rare cell binding paramagnetic particles.

17. The device of claim 14 further comprising the movement mechanism having a helical motion of the magnetic field relative to the receptacle.

18. The device of claim 14 wherein the movement mechanism further comprises a mechanism for rotating the electromagnet and one that translates the receptacle along an axis.

19. The device of claim 14 further comprising a video camera in a fixed location relative to the magnetic field, to provide visualization during motion.

20. The device of claim 14 further comprising a computing device with a non-transitory computer readable medium programmed for control of the device.
